(19) United States
(12) Patent Application Publication
(10) Pub. No.: US 2002/0035316 A1
(43) Pub. Date: Mar. 21, 2002

(54) Patient analysis and risk reduction system and associated methods

(75) Inventor: Robert A. Drazen, Longwood, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: HEALTHEHEART, INC., Altamonte Springs, FL

(21) Appl. No.: 09/943,509

(22) Filed: Aug. 30, 2001

Related U.S. Application Data
(60) Provisional application No. 60/229,266, filed on Aug. 30, 2000.

(51) Int. Cl. A61B 5/00

(52) U.S. Cl. 600/300; 128/920

(57) ABSTRACT

A patient analysis and risk reduction system includes a database for storing a plurality of different medical guidelines for different health conditions, such as cardiovascular disease. A processing device collects patient information from a user via the global network, and includes a risk evaluator for evaluating the patient information and generating a risk report based upon at least one of the different medical guidelines. A risk reduction unit evaluates the patient information and generates a physician's patient treatment plan including patient-specific recommendations for reducing risk based upon the different medical guidelines. A patient handout generator generates patient-specific instructions and educational material including guidelines for exercise, diet and lifestyle changes based upon the patient information, the risk report and the physician's patient treatment plan.

### RELATED APPLICATIONS

This application is based upon and claims priority from copending provisional application Serial No. 60/229,266 filed Aug. 30, 2000, the disclosure of which is incorporated by reference herein in its entirety.

### FIELD OF THE INVENTION

The present invention relates to health care, and, more particularly, to computerized medical systems and methods for providing patient risk assessment and/or medical diagnosis using patient information and medical guidelines.

### BACKGROUND OF THE INVENTION

Diagnostic systems, otherwise known as “expert systems” attempt to determine a cause as being the production of a plurality of events. Computer based diagnostic/expert systems are commonplace today and are applied to diagnosing problems in many different areas. For example, such systems are utilized to diagnose diseases, to locate geological formations, and to manage complex systems such as nuclear power plants, communications networks, etc. In medical terminology, a diagnostic/expert system attempts to determine the identity of a disease as being the production of two or more contemporaneous symptoms.

Expert systems are built around a knowledge base of specific information and an inference or rules engine. When an expert system is presented with a problem to solve, the rules engine combines information in the knowledge base with information about the problem. The rules engine applies its particular methodology to derive conclusions on the basis of the information provided. In such a system the knowledge base is made up of a set of condition/action rules in the form “if . . . then” or “yes . . . no”. A problem is presented to the system in the form of a set of true propositions (e.g. information obtained from the user). The system searches for rules which could satisfy a hypothesis and scans current conditions to determine whether the rule can be applied.

Disease management systems are expert systems that use a particular rules engine and knowledge base to automate the diagnosis and/or treatment of a specific disease or condition. For example, U.S. Publication No. 2001/0012913A1 to Iliff and entitled “Disease Management System and Method Including Correlation Assessment” is directed to a system and method for providing patient access to a an automated system for managing a specific health problem. However, the system attempts to take the practice of medicine out of the hands of physicians and put it into the hands of patients.

Another example of a medical expert system is U.S. Pat. No. 6,188,988 to Barry et al. and entitled “Systems, Methods and Computer Program Products for Guiding the Selection of Therapeutic Treatment Regimens.” This system is primarily concerned with guiding the user to select therapeutic regimens for a known disease such as HIV infection. This system is not concerned with determining and reducing a patient's risk relating to a certain health condition.

Also, the number of accepted and standardized medical practice guidelines for different health conditions relating to a certain condition, such as cardiovascular disease, are increasingly becoming difficult for the physician to manage and assimilate. Being capable of efficiently managing these guidelines while analyzing patient information to identify and reduce patient risk would reduce the cost of health care such as hospital stays and follow up care.

### SUMMARY OF THE INVENTION

In view of the foregoing background, it is therefore an object of the invention to provide a system and method for efficiently and accurately managing a plurality of medical guidelines while analyzing patient information to identify and reduce patient risk for a specific health condition.

This and other objects, features and advantages in accordance with the present invention are provided by a patient analysis and risk reduction system for use on a global network, such as the Internet. A database stores a plurality of different medical guidelines for different health conditions, such as cardiovascular disease, and a processing device collects patient information from a user via the global network. The processing device includes a risk evaluator for evaluating the patient information and generating a risk report based upon at least one of the different medical guidelines, a risk reduction unit for evaluating the patient information and generating a physician's patient treatment plan including patient-specific recommendations for reducing risk based upon the different medical guidelines, and a patient handout generator generates patient-specific instructions and educational material including guidelines for exercise, diet and lifestyle changes based upon the patient information, the risk report and the physician's patient treatment plan.

An electronic medical record (EMR) generator may be provided for generating a patient EMR based upon the patient information, the risk report and the physician's patient treatment plan. Also, a patient information database may store the patient information, the risk report and the physician's patient treatment plan. The processing device may monitor the patient information over time and update the risk report and the physician's patient treatment plan. Furthermore, the patient information may comprise gender, age, body mass index (BMI), cholesterol, blood pressure, blood sugar, allergies, diseases, family disease history, symptoms, lifestyle information, and current medications.

The patient handout generator may also generate disease-specific educational material, and the different medical guidelines preferably comprise medical guidelines for hypertension, diabetes, cholesterol, obesity and coronary disease. The patient-specific instructions and educational material may include guidelines for hypertension, diabetes, smoking cessation, weight management, nutrition and diet, cholesterol management and stress management. The physician's patient treatment plan may include links and references to the different medical guidelines used by the risk reduction unit.

The database may include a medication database, and the physician's patient treatment plan may include medication details and options including contraindications. Also, the risk reduction unit may include a user customizable evaluation module for evaluating the patient data and generating customized patient-specific recommendations for reducing risk.

Objects, features and advantages in accordance with the present invention are also provided by a method for analyzing patients and reducing risk using a global network and including storing a plurality of different medical guidelines for different health conditions, collecting patient information from a user via the global network, and evaluating the patient information and generating a risk report based upon at least one of the different medical guidelines. Also, the method includes evaluating the patient information and generating a physician's patient treatment plan including patient-specific recommendations for reducing risk based upon the different medical guidelines, and generating patient-specific instructions and educational material including guidelines for exercise, diet and lifestyle changes based upon the patient information, the risk report and the physician's patient treatment plan.

### BRIEF DESCRIPTION OF THE DRAWINGS

FIG. 1 is a schematic diagram of the system of the present invention connected to a global computer network.

FIG. 2 is a schematic diagram illustrating the details of the system of the present invention.

FIG. 3 illustrates a user interface for collecting patient information used by the system of FIG. 2.

FIGS. 4A-4C are flow charts illustrating an example of the risk evaluation performed by the system of FIG. 2.

FIG. 5 illustrates an example of a risk report generated by the system of FIG. 2.

FIG. 6 is a schematic diagram illustrating the details of an embodiment of the risk reduction unit of the system of FIG. 2.

FIGS. 7A and 7B are flow charts illustrating an example of the analysis performed by the risk reduction unit of FIG. 6.

FIGS. 8A and 8B illustrate an example of a physician's treatment plan generated by the risk reduction unit of FIG. 6.

### DETAILED DESCRIPTION OF THE PREFERRED EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

As will be appreciated by those skilled in the art, the present invention may be embodied as a method, data processing system, or computer program product. Accordingly, the present invention may take the form of an entirely hardware embodiment, an entirely software embodiment, or an embodiment combining software and hardware aspects. Furthermore, the present invention may be a computer program product on a computer-usable storage medium having computer readable program code on the medium. Any suitable computer readable medium may be utilized including, but not limited to, static and dynamic storage devices, hard disks, optical storage devices, and magnetic storage devices.

The present invention is described below with reference to flowchart illustrations of methods, systems, and computer program products according to an embodiment of the invention. It will be understood that each block of the flowchart illustrations, and combinations of blocks in the flowchart illustrations, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, implement the functions specified in the flowchart block or blocks.

These computer program instructions may also be stored in a computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

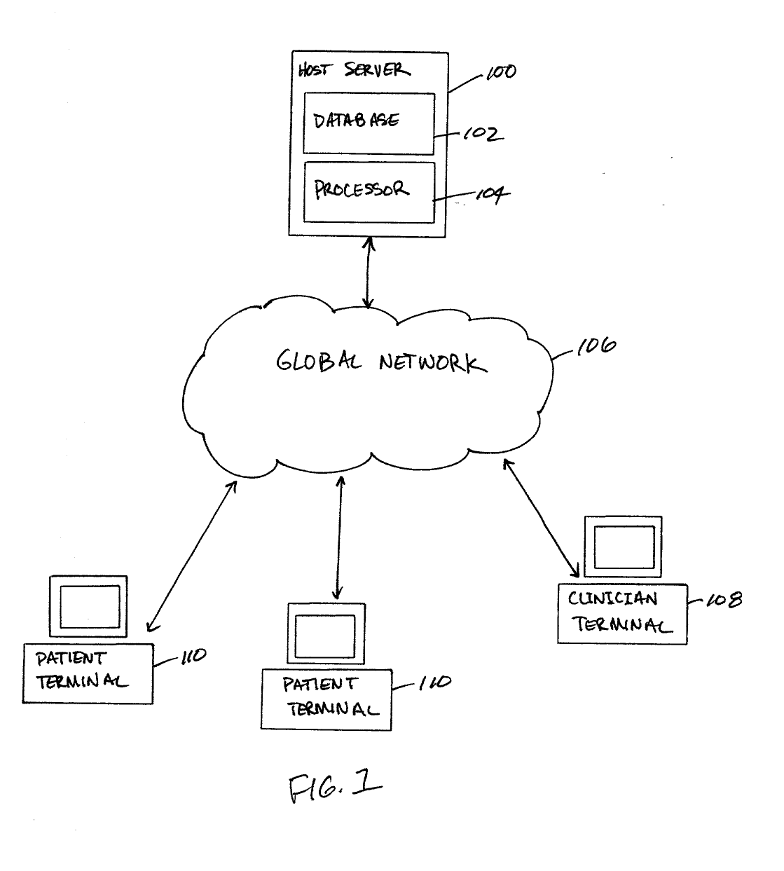

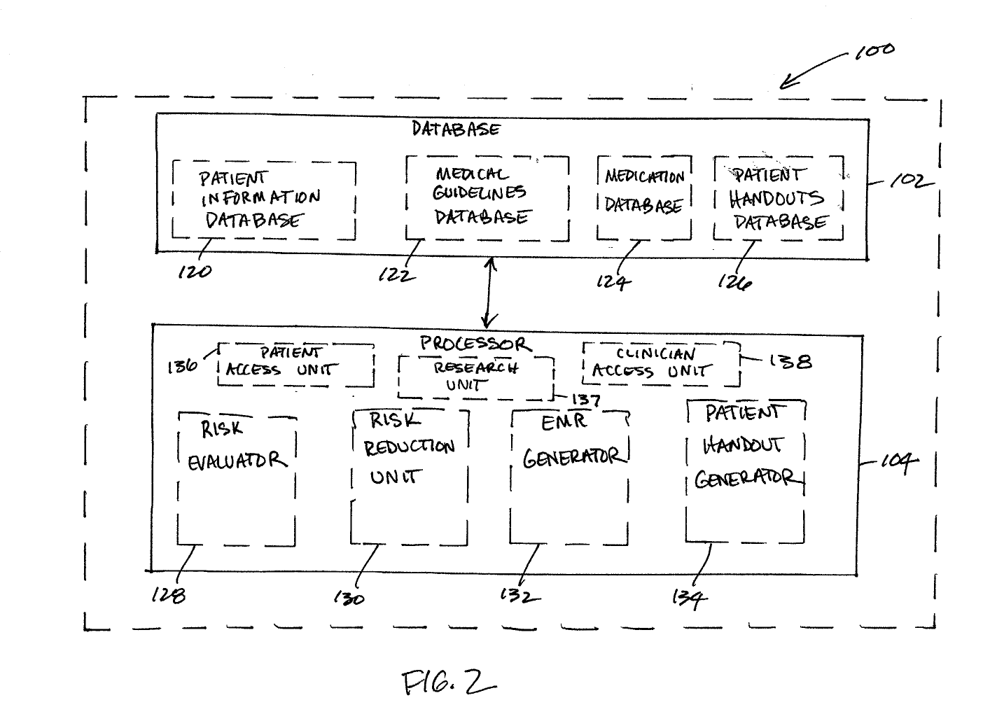

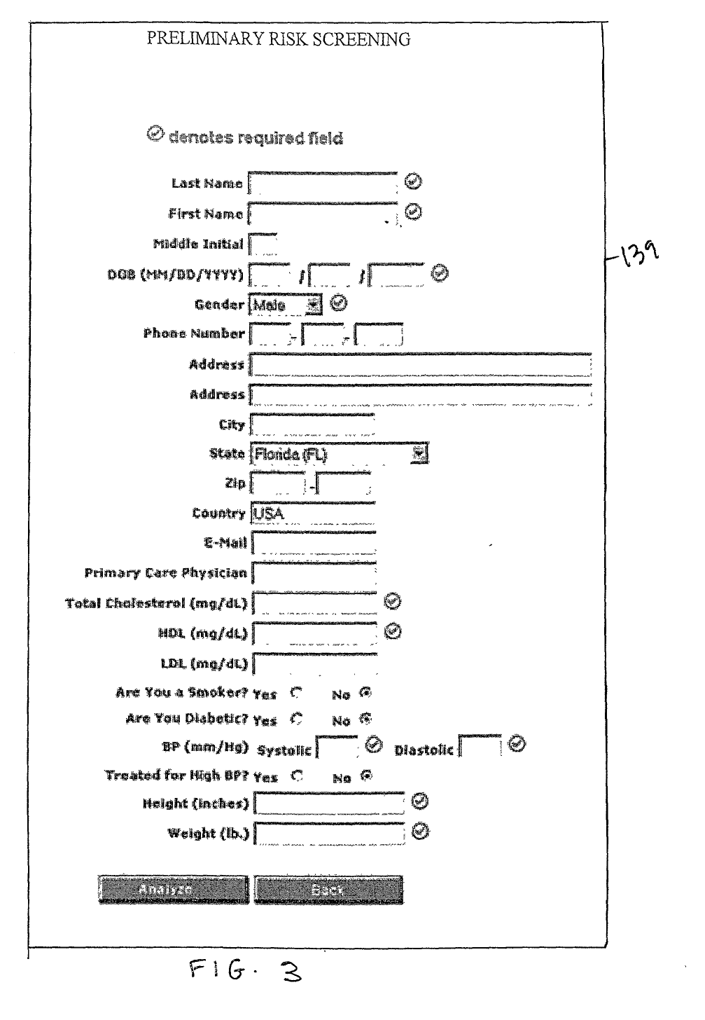

Referring to FIGS. 1 and 2, a patient analysis and risk reduction system 100 for use on a global network 106 will now be described. The global network may be an intranet, local area network (LAN) or wide area network (WAN), for example. However, for the present invention, the global network 106 is preferably the Internet, and the system 100 is preferably implemented as an Application Service Provider (ASP) model utilizing the functionality of the Internet. As such, the system 100 provides a comprehensive disease management methodology delivered to a physician/clinician's office 108 through the ASP. Accordingly, there is no need to install on-site software and all that is necessary to access the system 100 from the clinician remote computer terminal is a web browser and an internet connection, as would be appreciated by the skilled artisan.

A database 102 at least stores a plurality of different medical guidelines for different health conditions, such as cardiovascular disease. The medical guidelines are based upon accepted and standardized national or international medical guidelines published by experts in a particular area of medicine, such as medical guidelines for hypertension, diabetes, cholesterol, obesity and coronary disease. For example, the medical guidelines for hypertension are set forth in the Sixth Report of the Joint National Committee on Prevention, Detection, Evaluation and Treatment of High Blood Pressure (JNC6) convened by the National Institute of Health and published in the Archives of Internal Medicine, Volume 157, page 2413-2446, 1997.

Other examples of medical guidelines include, and are not limited to:

The Executive Summary of the Clinical Guidelines on the Identification, Evaluation and Treatment of Overweight and Obesity in Adults, published by the Expert Panel on the Identification, Evaluation and Treatment of Overweight and Obesity in Adults, convened by the National Institute of Health and published in the Archives of Internal Medicine, Volume 158, page 1855-1867, 1998;

The Smoking Cessation Clinical Practice Guideline, Number 18, published by the Agency for Health Care Policy and Research, April 1996, publication number 96-0692;

The Second Report from the Expert Panel on the Detection, Evaluation and Treatment of High Blood Cholesterol in Adults by the National Cholesterol Education Program, published by NIH in the U.S. Dept. of Health and Human Services, NIH publication number 93-3095, September 1993;

The 27^{th} Bethesda Conference, Matching the Intensity of Risk Factor Management with Hazards of Coronary Disease, published in the Journal of American College of Cardiology, Volume 27, pages 957-1047, April 1996, endorsed by the American College of Cardiology and the American Heart Association;

The Consensus Panel Statement, Preventing Heart Attack Deaths in Patients with Coronary Artery Disease, published by the American Heart Association, Volume 92, pages 2-4, 1995;

“A Global Measure of Perceived Stress,” Cohen and Karmarck, Journal of Health and Social Behavior, Volume 24, pages 385-396, 1983;

Standards of Medical Care for Patients with Diabetes Mellitus, published by the American Diabetes Association in Diabetes Care, Volume 21, Supplement One, January 1998, pages F23-F31; and

Diabetes Medical Practice Guidelines by the State of Florida Agency for Health Care Administration in consultation with the Diabetes Practice Guideline Advisory Committee, published by the state of Florida, January 1998.

The database 102 may include a patient information database 120 for storing patient data, a medical guidelines database 122 for storing the medical guidelines, a medication database 124 for storing information on medication including details, options, indications and contraindications, and a patient handout database 126 for storing educational material including guidelines for exercise, diet and lifestyle changes. The patient information database 120, medical guidelines database 122, medication database 124, and patient handout database 126 are illustrated as separate blocks of the database 102 for ease of understanding; however, it is understood that the information may be combined and accessed via associated data addresses as would be readily apparent to those skilled in the art. Furthermore, the patient information database 120 may be a research database for storing historical patient information data for a plurality of patients.

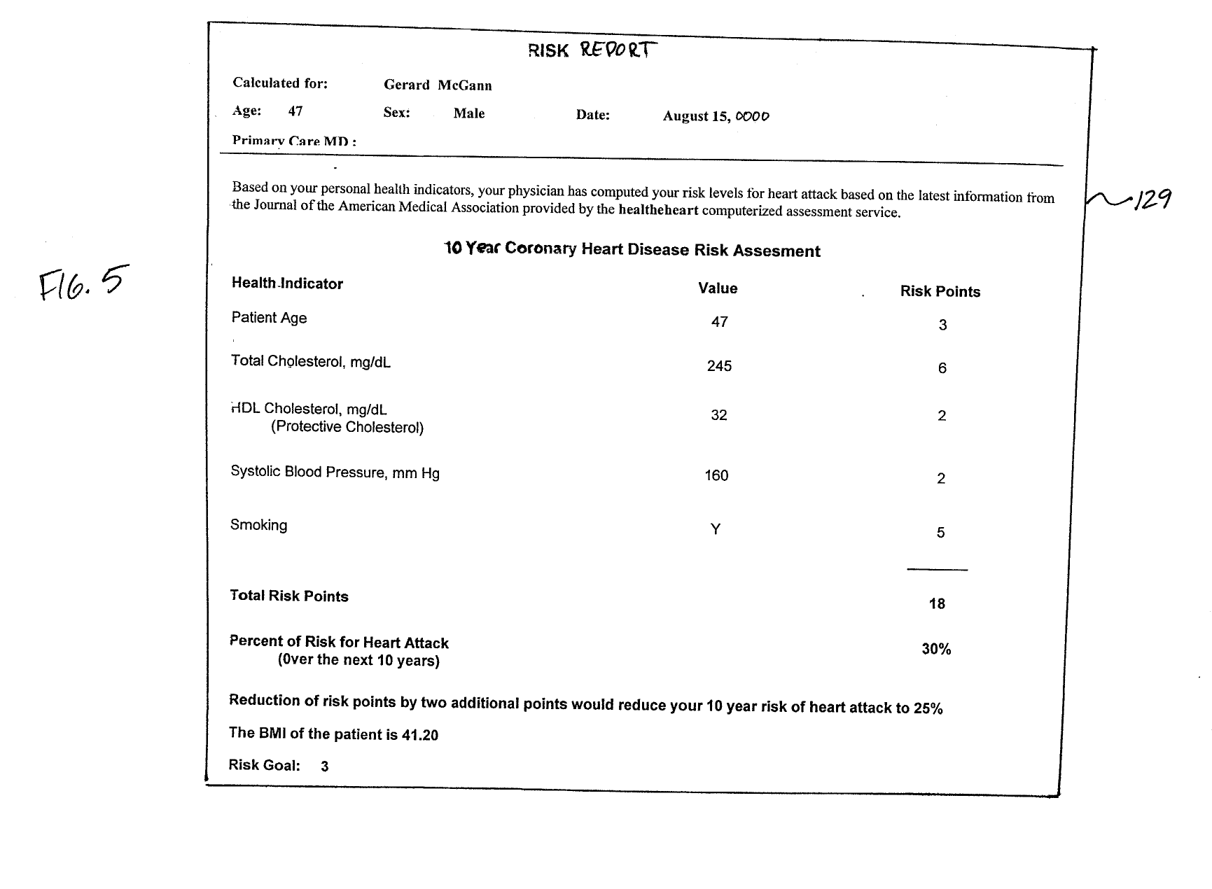

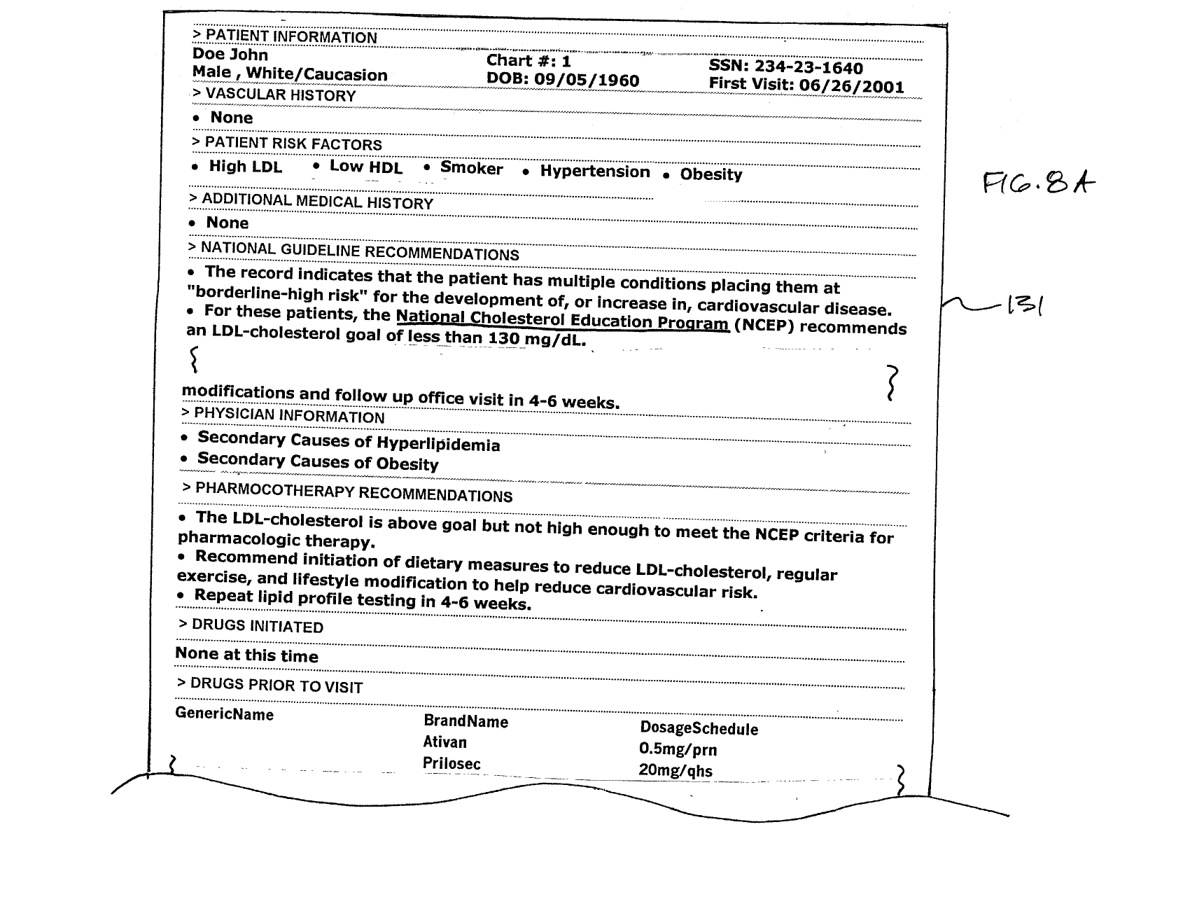

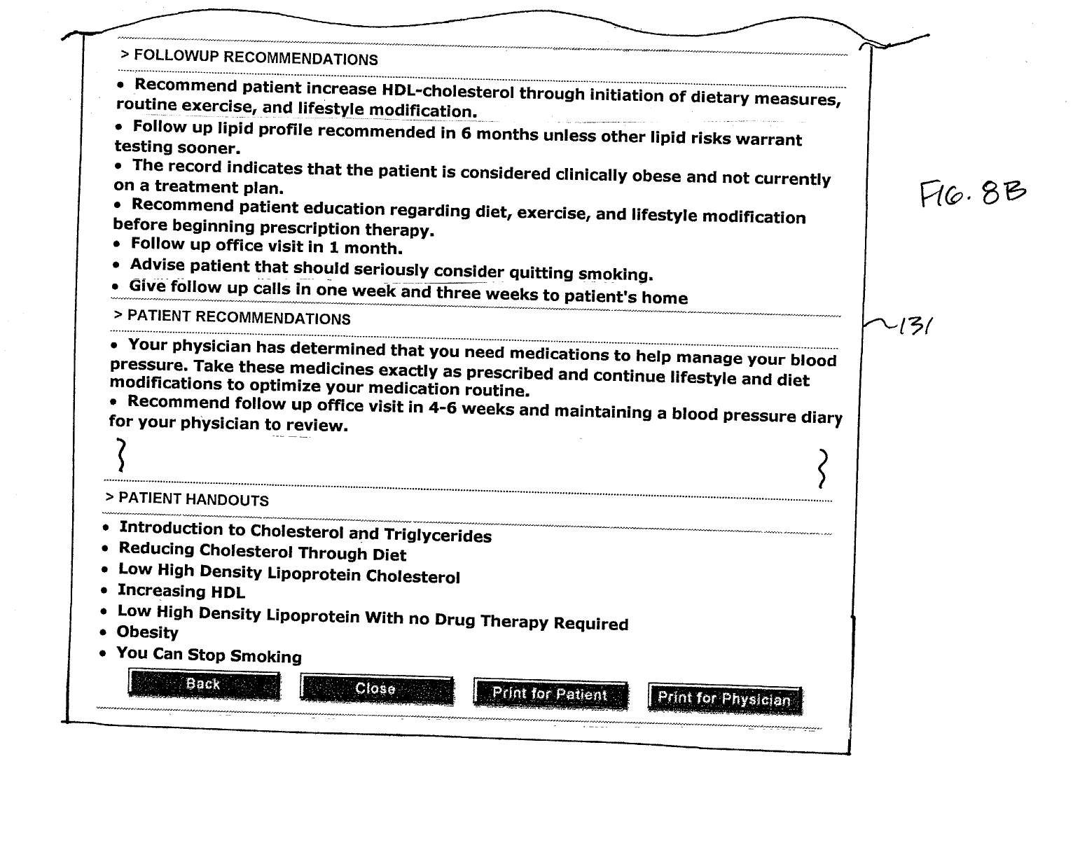

A processor 104 collects patient information from a user via the global network 106. As discussed, the user is typically a clinician using a remote computer terminal 108 connected to the system 100 via the Internet. The processor 104 includes a risk evaluator 128 for evaluating the patient information and generating a risk report 129 (FIG. 5) based upon at least one of the different medical guidelines, as will be discussed in greater detail below. Also, a risk reduction unit 130 is for evaluating the patient information and generating a physician's patient treatment plan 131 (FIGS. 8A and 8B) as will also be discussed in greater detail below. Such a treatment plan 131 includes patient-specific recommendations for reducing risk based upon the different medical guidelines.

A patient handout generator 134 generates patient-specific instructions and educational material including guidelines for exercise, diet and lifestyle changes based upon the patient information, the risk report 129 and the physician's patient treatment plan 131. The patient handout generator 134 uses the information stored in the patient handout database 126. An electronic medical record (EMR) generator 132 may be provided for generating a patient EMR based upon the patient information, the risk report 129 and the physician's patient treatment plan 131. EMR's are known in the art and require no further description herein. Of course, other records, such as progress notes, may be generated for the patient's chart or file.

A research unit 137 provides access to an authorized user of the system 100 via a remote computer terminal 108, and correlates historical patient information data and patient compliance with the physician's patient treatment plan 131 to generate outcome-specific research data. The outcome-specific research data may be used in clinical studies to evaluate and update the medical guidelines, for example. The outcome-specific research data may include health trends, and the risk reduction unit 130 may generate the physician's patient treatment plan 131 based upon the health trends.

Furthermore, a patient access unit 136 permits patient monitored information to be entered by an authorized patient using a remote computer terminal 110 with a secure connection to the system 100 via the global network 106. The patient monitored information is stored in the patient information database 120 and preferably includes daily blood pressure and blood sugar levels monitored at home by the patient. A clinician access unit 138 permits patient reported information and clinician recorded information to be entered by an authorized clinician using a remote computer terminal 108 with a secure connection to the system 100 via the global network 106. The patient reported information and clinician recorded information may be stored in the patient information database 120, and preferably comprises gender, age, body mass index (BMI), cholesterol, blood pressure, blood sugar, allergies, diseases, family disease history, symptoms, lifestyle information, and current medications.

The patient access unit 136 may also provide access to the patient-specific instructions and educational material, which may be guidelines for hypertension, diabetes, smoking cessation, weight management, nutrition and diet, cholesterol management and stress management.

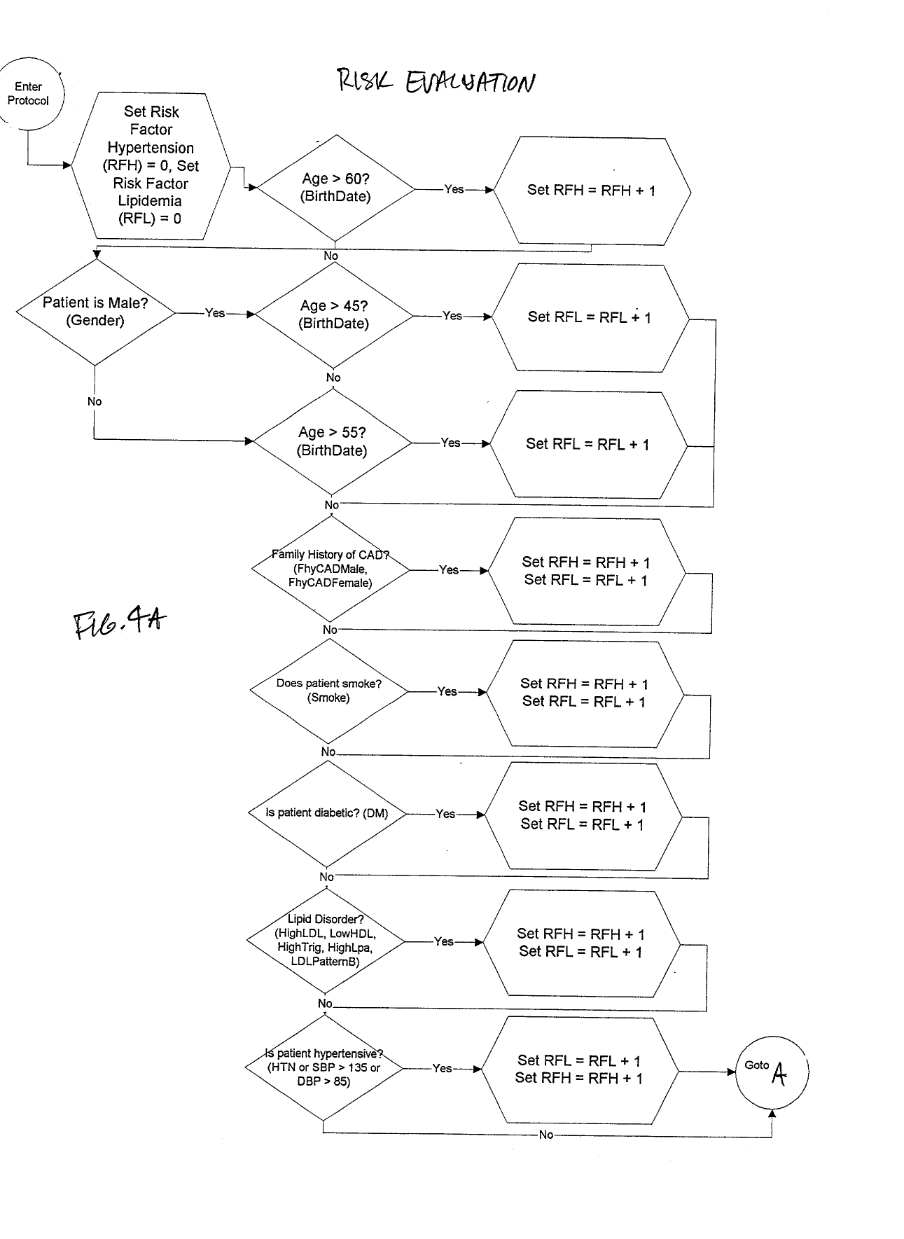

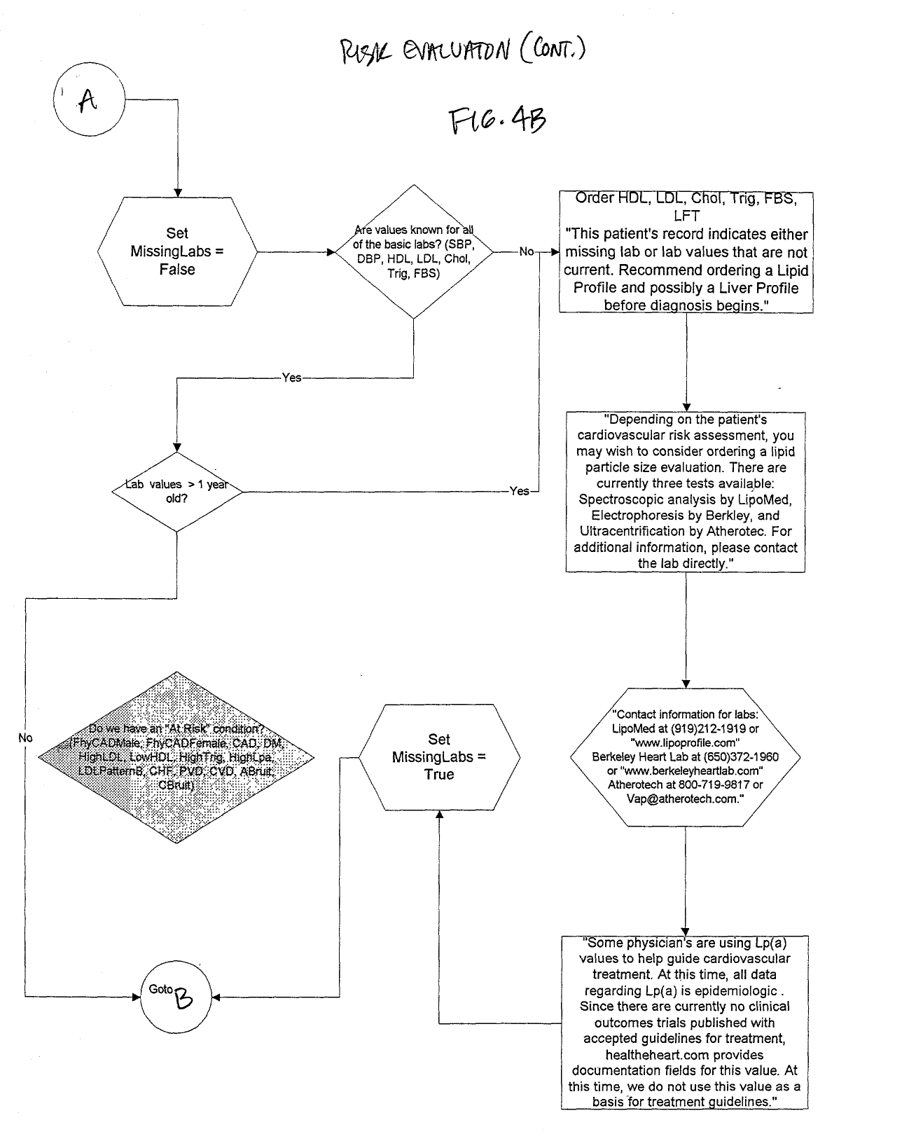

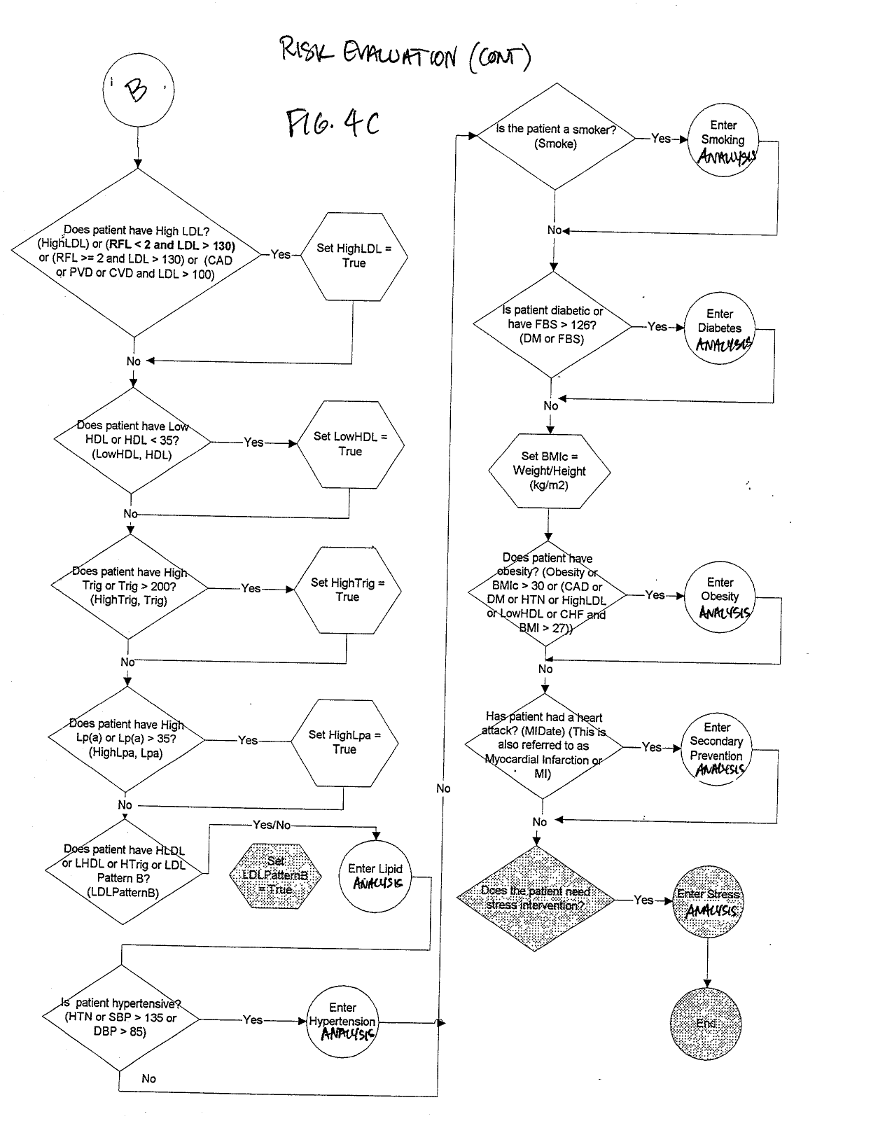

Referring to FIG. 3, an example of a user interface 139 accessible via a web browser on one of the computer terminals 108/110 is shown. The data fields for blood pressure and blood sugar may be updatable by both the authorized clinician and authorized patient while the other data fields may only be updated by the authorized clinician. After the patient information is entered, the risk evaluator 128 may operate to perform a risk evaluation, e.g. the risk of cardiovascular disease, for the patient. Referring to FIGS. 4A-4C, a flow chart illustrating an example of the risk evaluation for cardiovascular disease is shown. The risk evaluation includes yes/no/goto logic as would be appreciated by the skilled artisan.

The risk evaluator 128 may then generate the patient risk report 129 as shown, for example, in FIG. 5. As can be seen from the risk report 129, risk points are assessed for various values of health indicators, such as age, cholesterol, blood pressure etc. Then an overall percent of risk of having a heart attack over the next 10 years is calculated. Such a risk report may be analyzed by the physician, printed for the patient and/or printed for the patient chart.

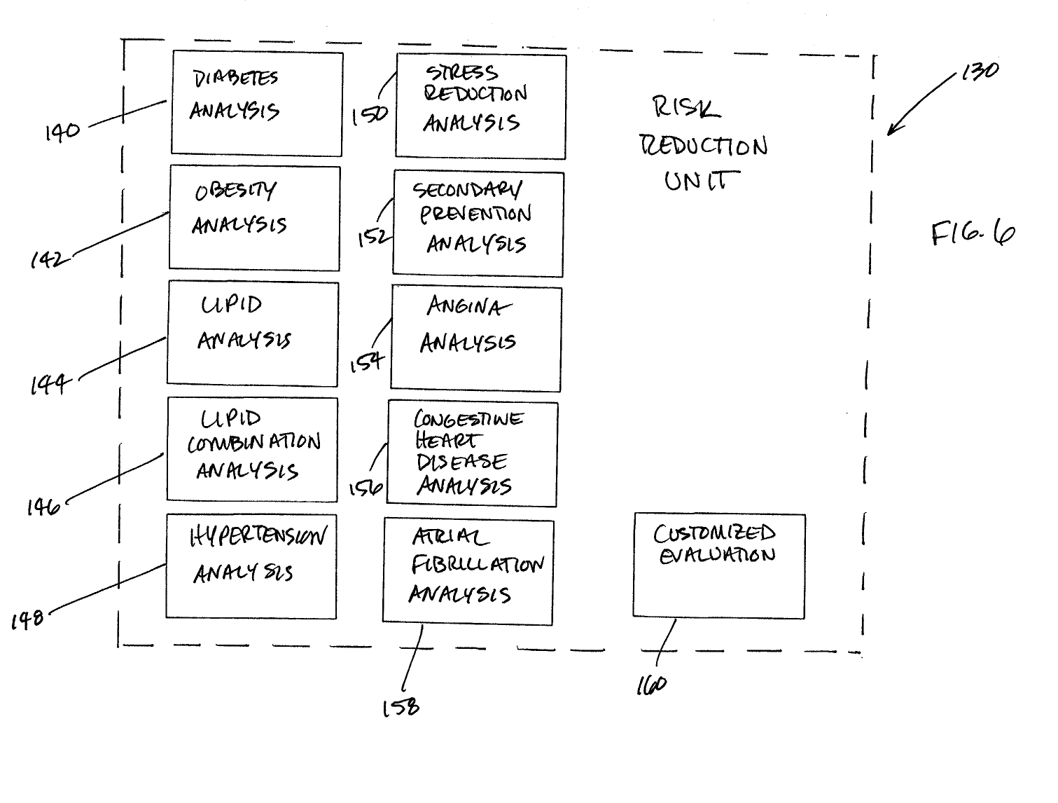

If the risk report 129 indicates a risk of disease which the physician believes is a concern for the patient's health, a treatment plan may be generated by the risk reduction unit 130. Of course, the system may also operate to automatically generate the treatment plan by the risk reduction unit 130 if any risk is indicated in the risk report 129 or by the risk evaluator 128. Referring to FIG. 6, the risk reduction unit 130 may include various analysis modules 140-158 which refer to and may be based upon the medical guidelines stored in the database 102 as discussed above. These analysis modules 140-158 correspond to different health conditions relating to the disease for which the patient is at risk. In this example, the disease is cardiovascular disease and the analysis modules include, but are not limited to, diabetes analysis 140, obesity analysis 142, lipid analysis 144, lipid combination analysis 146, hypertension analysis 148, stress reduction analysis 150, secondary prevention analysis 152, angina analysis 154, congestive heart disease analysis 156 and atrial fibrillation analysis 158.

Also, the risk reduction unit 130 may include a user customizable evaluation module 160 for evaluating the patient data and generating customized patient-specific recommendations for reducing risk. For example, if a particular physician wanted to vary his treatment plan for patients with specific conditions, the customizable evaluation module 160 may implement that physician's variations for one or more of the medical guidelines.

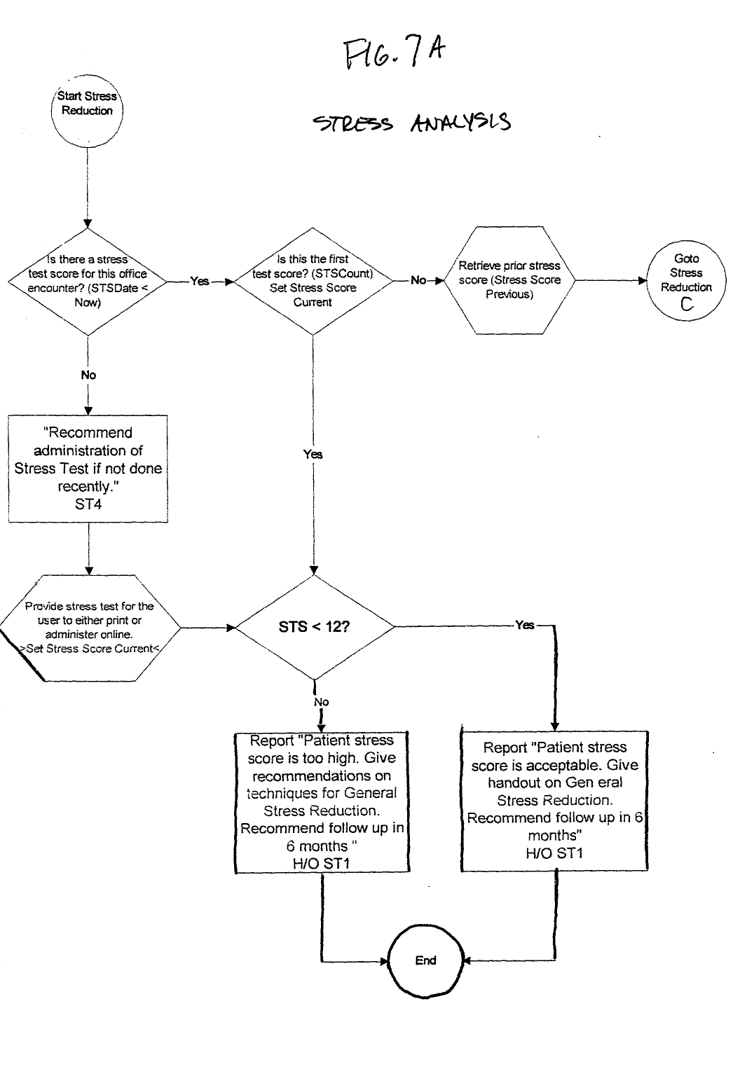

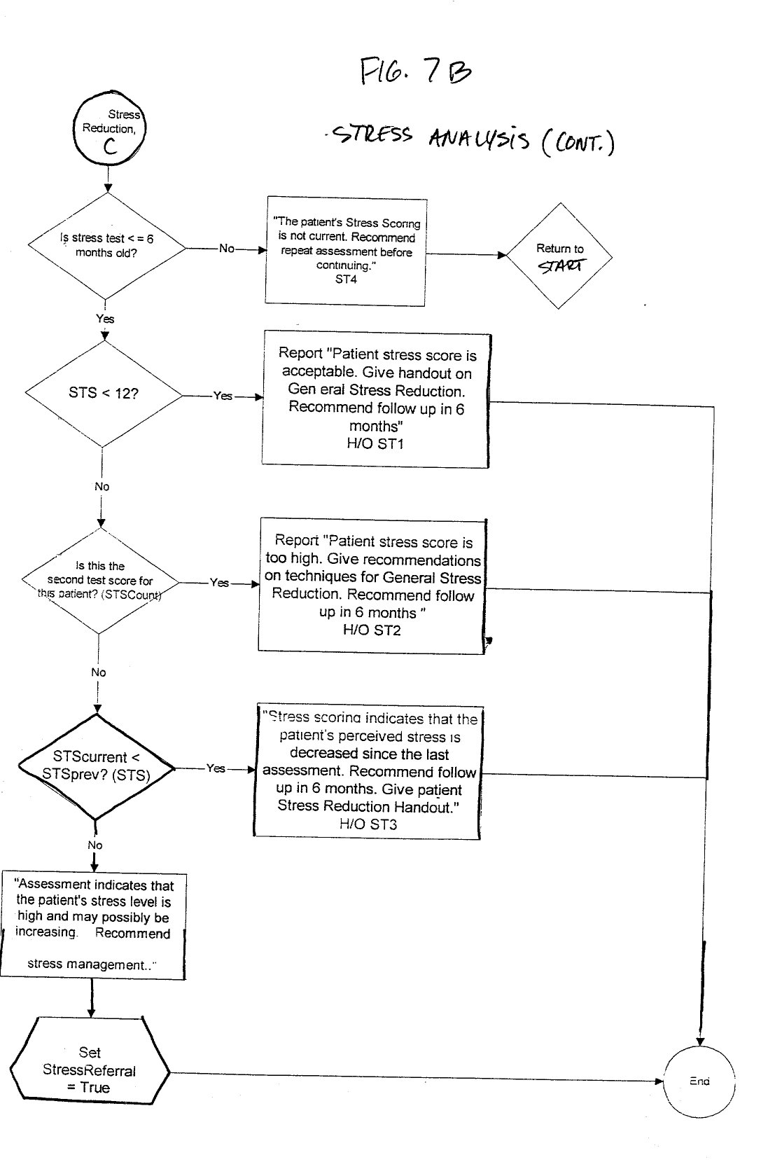

An example of the operation of an analysis module is illustrated in the flow chart of FIGS. 7A-7B. Specifically, an example of the stress reduction analysis module 150 which may be based upon and/or refers to “A Global Measure of Perceived Stress,” Cohen and Karmarck, Journal of Health and Social Behavior, Volume 24, pages 385-396, 1983, is shown. Of course the other analysis modules 140-158 may be similarly implemented with yes/no or if/then rules which evaluate the patient information.

Referring now to FIGS. 8A and 8B, a physician's patient treatment plan 131 may be generated by the risk reduction unit 130 after the patient information has been evaluated. Such a treatment plan 131 preferably includes at least some of the patient information collected by the processor 104, medical guideline recommendations, information for the physician, medication information, follow-up recommendations, patient instructions and/or links to patient handouts. The patient handout list may include links to digital versions or hard copies of the handouts which are generated by the patient handout generator 134.

The patient information database 120 may store the patient information, the risk report 129 and the physician's patient treatment plan 131. In such a case, the processor 104 may monitor the patient information over time and update the risk report 129 and the physician's patient treatment plan 131 accordingly.

The patient handout generator 134 may also generate disease-specific educational material. The patient-specific instructions and educational material may include guidelines for hypertension, diabetes, smoking cessation, weight management, nutrition and diet, cholesterol management and stress management as discussed above. The physician's patient treatment plan 131 may include active links and/or references to the different medical guidelines used by the risk reduction unit. The physician's patient treatment plan 131 may include medication details and options including contraindications.

A method aspect of the invention includes a method for analyzing patients and reducing risk using a global network 106 and including storing a plurality of different medical guidelines for different health conditions, collecting patient information from a user via the global network, and evaluating the patient information and generating a risk report 129 based upon at least one of the different medical guidelines. Also, the method includes evaluating the patient information and generating a physician's patient treatment plan 131 including patient-specific recommendations for reducing risk based upon the different medical guidelines, and generating patient-specific instructions and educational material including guidelines for exercise, diet and lifestyle changes based upon the patient information, the risk report 129 and the physician's patient treatment plan 131.

Furthermore, the method may include storing historical patient information data for a plurality of patients in the patient information database or research database 120, and correlating historical patient information data and patient compliance with the physician's patient treatment plan 131 to generate outcome-specific research data, such as health trends. Subsequently generated patient treatment plans 131 may also be based upon the health trends.

Also, the method may include storing patient monitored information, entered by a patient using a first remote computer 110 via the global network 106, in a patient information database 120, and storing patient reported information and clinician recorded information, entered by a clinician using a second remote computer 108 via the global network, in the patient information database. Permitting access to the patient-specific instructions and educational material by the patient using the first remote computer 110 via the global network 106 may also be provided.

A patient electronic medical record (EMR) based upon the patient information, the risk report and the physician's patient treatment plan is also preferably generated. Moreover, the method may include monitoring the patient information over time and updating the risk report 129 and the physician's patient treatment plan 131 based upon updated patient information.

In sum, the invention is preferably embodied as a core software application that operates from a remote personal computer 108 connected to a global network 106, such as the Internet, and supports physicians by giving them immediate access to a wide and deep range of pertinent data and information at the point of care. The application, run by an underlying rules engine application that enables convergence of data, takes seemingly disparate patient data and hunts for risk associated with, for example, cardiovascular disease. The system is capable of bringing order, standardization and consistency to a wide range of healthcare businesses responsible for disease management and cost containment, and is ideal for physicians and their extenders working in busy practices in clinical, hospital and community-based settings. Accessing the web-enabled application on a standard PC desktop during the patient visit, the physician or physician extender begins building an electronic medical record (EMR) for each patient. The program keeps track of pertinent patient information, such as lab values, family history, patient demographics, drug therapy, and the details of the physician-patient encounter.

Through the collection of this data, the system can then measure patient outcomes, track patient compliance, document the encounter, and include information on specific patient education materials given to the patient. Later, the physician can query the database for specific data such as blood pressure variations, pertinent patient history, and so forth. The system also measures outcomes so that the physician can better track the progress of a patient following a certain protocol to determine whether the treatment is effective.

The system and method facilitates standardization or “reproducibility” of the treatment planning process with options for personalization. The treatment plan produced by the application is comprehensive, containing patient-specific recommendations for medical care and follow-up. In addition, it incorporates patient education materials addressing dietary and exercise recommendations, important medication-related instructions and other information that facilitates self-care and compliance. Copies of the resulting documents may be placed in the patient's medical record, thereby enhancing physician documentation of the patient encounter.

The invention harnesses technology to streamline and optimize disease detection and management processes while ensuring that the power to oversee and individualize patient care stays in the hands of the doctor. The application goes beyond traditional management of disease by incorporating both prevention and detection with a strong emphasis on early risk identification. This system and method was designed with doctors and their extenders in mind.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A cardiovascular analysis system for use on a global network comprising:
a database for storing a plurality of different medical guidelines for different health conditions relating to cardiovascular disease; and
a processing device connected to the database and for collecting patient information from a user via the global network, the processing device comprising
a risk evaluator for evaluating the patient information and generating a cardiovascular risk report based upon at least one of the different medical guidelines,
a risk reduction unit for evaluating the patient information and generating a physician's patient treatment plan including patient-specific recommendations for reducing cardiovascular risk based upon the different medical guidelines,
an electronic medical record (EMR) generator for generating a patient EMR based upon the patient information, the cardiovascular risk report and the physician's patient treatment plan, and
a patient handout generator for generating patient-specific instructions and educational material including guidelines for at least one of exercise, diet and lifestyle changes based upon the patient information, the cardiovascular risk report and the physician's patient treatment plan.

2. A cardiovascular analysis system according to claim 1 wherein the database includes a patient information database for storing the patient information, the cardiovascular risk report, the patient EMR and the physician's patient treatment plan.

3. A cardiovascular analysis system according to claim 2 wherein the processing device monitors the patient information over time and updates the cardiovascular risk report and the physician's patient treatment plan.

4. A cardiovascular analysis system according to claim 1 wherein the patient information comprises at least one of gender, age, body mass index (BMI), cholesterol, blood pressure, allergies, diseases, family disease history, symptoms, lifestyle information, and current medications.

5. A cardiovascular analysis system according to claim 1 wherein the patient handout generator also generates disease-specific educational material.

6. A cardiovascular analysis system according to claim 1 wherein the different medical guidelines comprise medical guidelines for hypertension, diabetes, cholesterol, obesity and coronary disease.

7. A cardiovascular analysis system according to claim 1 wherein the patient-specific instructions and educational material includes guidelines for at least one of hypertension, diabetes, smoking cessation, weight management, nutrition and diet, cholesterol management and stress management.

8. A cardiovascular analysis system according to claim 1 wherein the physician's patient treatment plan includes links to the different medical guidelines used by the risk reduction unit.

9. A cardiovascular analysis system according to claim 1 wherein the physician's patient treatment plan includes references to the different medical guidelines used by the risk reduction unit.

10. A cardiovascular analysis system according to claim 1 wherein the database includes a medication database.

11. A cardiovascular analysis system according to claim 10 wherein the physician's patient treatment plan includes medication details and options including contraindications.

12. A cardiovascular analysis system according to claim 1 wherein the risk reduction unit comprises a user customizable evaluation module for evaluating the patient data and generating customized patient-specific recommendations for reducing cardiovascular risk.

13. A patient analysis and risk reduction system for use on a global network comprising:
a database for storing a plurality of different medical guidelines for different health conditions; and
a processing device associated with the database and for collecting patient information from a user via the global network, the processing device comprising
a risk evaluator for evaluating the patient information and generating a risk report based upon at least one of the different medical guidelines,
a risk reduction unit for evaluating the patient information and generating a physician's patient treatment plan including patient-specific recommendations for reducing risk based upon the different medical guidelines, and
a patient handout generator for generating patient-specific instructions and educational material including guidelines for at least one of exercise, diet and lifestyle changes based upon the patient information, the risk report and the physician's patient treatment plan.

14. A patient analysis and risk reduction system according to claim 13 wherein the processing device further comprises an electronic medical record (EMR) generator for generating a patient EMR based upon the patient information, the risk report and the physician's patient treatment plan.

15. A patient analysis and risk reduction system according to claim 13 wherein the database includes a patient information database for storing the patient information, the risk report and the physician's patient treatment plan.

16. A patient analysis and risk reduction system according to claim 15 wherein the processing device monitors the patient information over time and updates the risk report and the physician's patient treatment plan.

17. A patient analysis and risk reduction system according to claim 13 wherein the patient information comprises at least one of gender, age, body mass index (BMI), cholesterol, blood pressure, blood sugar, allergies, diseases, family disease history, symptoms, lifestyle information, and current medications.

18. A patient analysis and risk reduction system according to claim 13 wherein the patient handout generator also generates disease-specific educational material.

19. A patient analysis and risk reduction system according to claim 13 wherein the different medical guidelines comprise medical guidelines for hypertension, diabetes, cholesterol, obesity and coronary disease.

20. A patient analysis and risk reduction system according to claim 13 wherein the patient-specific instructions and educational material includes guidelines for at least one of hypertension, diabetes, smoking cessation, weight management, nutrition and diet, cholesterol management and stress management.

21. A patient analysis and risk reduction system according to claim 13 wherein the physician's patient treatment plan includes links to the different medical guidelines used by the risk reduction unit.

22. A patient analysis and risk reduction system according to claim 13 wherein the physician's patient treatment plan includes references to the different medical guidelines used by the risk reduction unit.

23. A patient analysis and risk reduction system according to claim 13 wherein the host database includes a medication database.

24. A patient analysis and risk reduction system according to claim 23 wherein the physician's patient treatment plan includes medication details and options including contraindications.

25. A patient analysis and risk reduction system according to claim 13 wherein the risk reduction unit comprises a user customizable evaluation module for evaluating the patient data and generating customized patient-specific recommendations for reducing risk.

26. A method for analyzing patients and reducing risk using a global network comprising:
storing a plurality of different medical guidelines for different health conditions;
collecting patient information from a user via the global network;
evaluating the patient information and generating a risk report based upon at least one of the different medical guidelines;
evaluating the patient information and generating a physician's patient treatment plan including patient-specific recommendations for reducing risk based upon the different medical guidelines; and
generating patient-specific instructions and educational material including guidelines for at least one of exercise, diet and lifestyle changes based upon the patient information, the risk report and the physician's patient treatment plan.

27. A method according to claim 26 further comprising generating a patient electronic medical record (EMR) based upon the patient information, the risk report and the physician's patient treatment plan.

28. A method according to claim 26 further comprising storing the patient information, the risk report and the physician's patient treatment plan in a patient information database.

29. A method according to claim 28 further comprising monitoring the patient information over time and updating the risk report and the physician's patient treatment plan.

30. A method according to claim 26 wherein the patient information comprises at least one of gender, age, body mass index (BMI), cholesterol, blood pressure, blood sugar, allergies, diseases, family disease history, symptoms, lifestyle information, and current medications.

31. A method according to claim 26 further comprising generating disease-specific educational material based upon the patient information, the risk report and the physician's patient treatment plan.

32. A method according to claim 26 wherein the different medical guidelines comprise medical guidelines for hypertension, diabetes, cholesterol, obesity and coronary disease.

33. A method according to claim 26 wherein the patient-specific instructions and educational material includes guidelines for at least one of hypertension, diabetes, smoking cessation, weight management, nutrition and diet, cholesterol management and stress management.

34. A method according to claim 26 wherein the physician's patient treatment plan includes links to the different medical guidelines used by the risk reduction unit.

35. A method according to claim 26 wherein the physician's patient treatment plan includes references to the different medical guidelines used by the risk reduction unit.

36. A method according to claim 26 further comprising storing medication information in a medication database.

37. A method according to claim 36 wherein the physician's patient treatment plan includes medication details and options including contraindications.

38. A method according to claim 26 wherein the different health conditions are cardiovascular related conditions, and the risk is cardiovascular disease.
